(19) United States
(12) Patent Application Publication
(10) Pub. No.: US 2015/0182622 A1
(43) Pub. Date: Jul. 2, 2015

(54) TREATMENT AND PREVENTION OF RETINAL INJURY AND SCARRING

(71) Applicant: THE SCHEPENS EYE RESEARCH INSTITUTE, Boston, MA (US)

(72) Inventor: Kameran Lashkari, Boston, MA (US)

(21) Appl. No.: 14/407,823

(22) PCT Filed: Jun. 14, 2013
(86) PCT No.: PCT/US13/45856

§ 371 Date: Dec. 12, 2014

Related U.S. Application Data
(60) Provisional application No. 61/659,645, filed on Jun. 14, 2012.

(51) Int. Cl. A61K 39/395

(52) U.S. Cl. CPC A61K 39/3955

(57) ABSTRACT

The present invention relates to a method for the prevention of scar formation and vision loss due to laser injuries caused by exposure to laser radiation. The method involves the administration to a subject exposed to laser radiation of an effective amount of a pharmaceutical composition containing an inhibitor of c-Met activity, such as an antibody to c-Met.

### CROSS-REFERENCE TO RELATED APPLICATION

This application is a national stage entry of PCT Application No. PCT/US13/45856, filed Jun. 14, 2013, and claims priority to U.S. Provisional Patent Application No. 61/659,645, filed Jun. 14, 2012, the disclosures of each of which are incorporated by reference herein in their entireties.

### GOVERNMENT SPONSORED RESEARCH OR DEVELOPMENT

This work may have been funded in whole or in part by a grant from the Department of Defense of the Federal Government. The Federal Government may have certain rights in the invention.

### BACKGROUND OF THE INVENTION

Lasers have been broadly applied in our world, and laser instruments are being increasingly employed in a vast variety of fields, including military, health, educational, and commercial laboratories. The use of lasers has increased many fold in the military, owing to the military's use of laser range finders, target designators and long distance communications. Even in the field of ophthalmology, the use of lasers has increased many fold. Along with this increase in the use of laser devices, there is also a proportionate increase in ocular exposure to laser radiation. A review of military and civilian data sources in 1997 estimated that 220 confirmed laser eye injuries occurred between 1964 and 1996.

Laser eye injuries often cause devastating disability and significant costs to the military in terms of medical care and lost work time. Exposure to lasers can cause severe clinical ocular injuries that mostly damage the retinal pigment epithelium (“RPE”) layer of the human eye by photothermal and photodisruptive mechanisms. These laser induced injuries can vary from scars as small as a few mm in size to full thickness macular formation, causing disruption of the foveal anatomy.

The clinical course of retinal laser injuries is characterized by initial blurred and distorted vision, possibly followed by severe late complications, which include fibrovascular scar formation, choriodal neovascularization, and central vision loss.

Apart from injury to retinal neurons due to direct exposure to lasers, there are also late onset complications that arise from the excessive wound healing after the initial insult. This can lead to overt fibrosis and granulation tissue formation beyond the original confines of the injured area (known as “creep”). Frequently secondary migration of the scar towards the foveal center can affect final visual recovery. This has been a therapeutic dilemma in the care management of soldiers who have received accidental laser injuries from Nd:YAG lasers. Limiting the size of the scar by controlling the wound enlargement and inhibiting aberrant RPE cell migration are critical factors in designing therapies for laser injury.

Hepatocyte growth factor (“HGF”), also known as “scatter factor”, was originally discovered and cloned as a potent mitogen for mature hepatocytes. HGF is predominantly expressed by cells of stromal origin, including fibroblasts, vascular smooth muscle cells and glial cells. Previous studies have indicated that HGF exhibits pleiotropic biological functions in its target cells as mitogen, motagen and morphogen, and also exhibits proangiogenic and anti-apoptotic properties. HGF is synthesized by mesenchyme-derived cells (namely fibroblasts), which primarily target epithelial cells in a paracrine manner through its receptor, c-Met.

As the only known specific receptor for HGF, c-Met, a receptor tyrosine kinase, mediates virtually all HGF-induced biological activities. c-Met is a 190 kDa product of the met proto-oncogene composed of a 45 kDa α-chain that is disulfide-linked to a 145 kDa β-chain. Stimulation of c-Met mediation by HGF results in receptor dimerization, which induces phosphorylation at 1349 and 1356 salient tyrosine sites and its kinase domain.

In the retina, c-Met is mainly expressed in RPE cells. In response to pathologic conditions, RPE cells initiate a post-injury process and become transformed from a stationary epithelial state to a spindle-shaped, migratory and proliferative mesenchymal state, leading to the transretinal membrane formation associated with the development of proliferative vitreoretinopathy (“PVR”). Excessive RPE layer injury response can further deteriorate visual outcome after laser-induced injury, leading to scar formation beyond the confines of the site of the injury itself, and usually towards the central macula.

In view of the persistence and frequency of laser eye injuries, both for military and civilian personnel, as well as a current lack of available treatment options, it will readily be appreciated that a need exists to improve the prevention and treatment of such injuries. This and other objectives of the invention will be clear from the following description.

### SUMMARY OF THE INVENTION

The invention is directed to a method of reducing scar formation and vision loss due to exposure to laser light by individuals in both the military and civilian sectors. A method for simulating laser induced injuries to the RPE in humans was devised in a mouse model. This model also served to evaluate the role of c-Met in the pathogenesis and progression of late stage complications of laser-induced RPE injuries, and to confirm the involvement of c-Met in the migration of RPE cells as an early response to injuries. Using this model, it was demonstrated that retinal laser injury increases the expression of both HGF and c-Met, and induces the phosphorylation of c-Met. It was also shown that the constitutive activation of c-Met induces more robust RPE migration while the abrogation of the receptor reduces these responses. c-Met was therefore identified as a potential therapeutic target influencing post-injury response to laser burns, and to control the aberrant RPE migration and wound enlargement after laser-induced injury.

Accordingly, in one embodiment, a method of reducing scar formation and vision loss comprises administering to a mammal, preferably a human, a pharmaceutical composition containing an active ingredient that inhibits the activity of the c-Met receptor. The pharmaceutical composition can be administered locally, topically, intraocularly, peribulbarly or intravitreally, depending on the desired route of administration.

In one aspect, the active ingredient in the pharmaceutical composition is an antibody that binds to the c-Met receptor, or an antagonist to the c-Met receptor. In this aspect, the activity of the c-Met receptor can be inhibited by interfering with the binding of c-Met to the ligand HGF.

In a further aspect, the scar format and vision loss are the result of the exposure of the eye to penetrating or non-penetrating ocular trauma, retinal detachment resulting in the release of RPE cells, choroidal scar formation, or a laser selected from the group consisting of thermal lasers, Nd:YAG lasers, and non-thermal lasers, such as therapeutic photodynamic lasers.

In a still further aspect, the scar formation and vision loss results from the migration of RPE cells into the outer retina of a human eye.

The present invention, accordingly, comprises the construction, combination of elements and components, and/or the arrangement of parts and steps which are exemplified in the following detailed disclosure. The foregoing aspects and embodiments of the invention are intended to be illustrative only, and are not meant to restrict the spirit and scope of the claimed invention.

### BRIEF DESCRIPTION OF THE DRAWINGS

The foregoing and other advantages and features of the invention will become apparent upon reading the following detailed description with reference to the accompanying figures and drawings.

FIG. 1 (1A-1C) is a structural diagram of cMet in TPR-Met mice (1A) and a schematic of Cre-mediated knock-out of c-Met by AAV-Cre delivered subretinally in the homozygous c-Met^{fl/fl} mice (1B). Photomicrographs show the results of AAV-Cre injection and AAV-GFP injection (1C). Before AAV-Cre injection, retina Iysates of c-Met^{fl/fl} mice were prepared for genotyping by PCR reaction. A 380 bp amplification fragment was specific to the floxed allele (a and c); a 300 bp fragment to the wild-type allele (e); in AAV-Cre injected mice, a 650 bp fragment was detected specific to the deleted allele (b, indicated by an arrowhead), while mice subretinally injected with AAV-GFP did not show the corresponding band (d).

FIG. 2 are a series of photomicrographs showing terminal deoxynucleotidyl transferase dUTP nick end labeling (TUNEL) indicating that laser injury induced early apoptosis in the outer nuclear layer (ONL) in B6 mice. TUNEL is a common method for detecting DNA fragmentation that results from apoptotic signaling cascades. No significant morphological disorganization was observed in the retina within hours after laser burns (A, D). Nuclei in ONL exhibited signs of apoptosis about 12 hr after laser injury (B-C) and reached the apoptosis peak by day 3 (E-F, TUNEL-positive nuclei labeling with fluorescence). The apoptotic and dead cells are indicated by arrowheads, respectively (G-I). Scale bar for images: 100 μm. Abbreviations (the same all FIGS.): B6 mice, C57BL/6 mice; INL, inner nuclear layer; ONL, outer nuclear layer; and RPE, retinal pigment epithelium.

FIG. 3 (3A-3F) are a series of photomicrographs showing representative images of morphological features in the retinal layer following laser burn injury in B6 mice. Tissue were embedded in paraffin and stained with hematoxylin and eosin. Eye receiving sham laser injury shows intact retina and RPE layers (A). At 12-24 hr after the laser burns, INL and ONL begin to show structural disorganization with some photoreceptor loss (B-C). RPE monolayer was disrupted and pigmented cells were observed in the subretinal space (arrow; C). On day 3, significant photoreceptor loss was observed in conjunction within the laser-injured area (D). No photoreceptor was found in the injury area at day 14 (E, indicated by arrows). The RPE monolayer reformed at the wounded area suggesting reformation of a new blood-retina barrier (F, indicated by arrows). The scale bar for all images: 100 μm. Abbreviation: GCL, ganglion cell layer.

FIG. 4 (4A-4D) are a series of graphs showing quantified gene expression in laser-injured retinas of B6 mice. Data are presented as a fold increase over sham-treated eyes, and normalized to the expression of GAPDH. mRNA expression of c-Met, the cognate receptor for HGF, reached its peak value around 12 hr after the laser injury (A), while the mRNA level of HGF peaked at 3 hr (B) (indicated by arrows in C, respectively). A hysteresis relationship was identified between the expression of c-Met and HGF (arrowheads, C). The expression of phosphorylated c-Met (p-Met) did not show any significant change over time after laser application (D). Abbreviations: HGF, hepatocyte growth factor; GAPDH, glyceraldehyde 3-phosphate dehydrogenase; Con, control retinas received sham laser burns; IHC, immunohistochemical staining. *P<0.05 (Mann-Whitney U test, n=5).

FIG. 5 (5A-5AA) are a series of photomicrographs with bar graphs showing the dynamic changes in the expression of c-Met, p-Met and RPE65 in the retina of B6 and TPR-Met mice after laser injury. After sham laser injury, very trace c-Met expression was detected in the control retina (A). c-Met expression was increased up to day 3 (B). On day 7 and 14, c-Met expression (C-D) decreased but remained higher than control (A). Similarly, in TPR-Met mice, c-Met expression significantly increased after laser injury (F) although obvious expression was found in sham-treated eyes (control; E). c-Met expression decreased from day 7 to day 14. On day 7, migrated c-Met positive cells were observed in the outer retina (C-D, G-H). On day 14, the disrupted RPE layer was reformed as a monolayer (D, H). Expression of (phosphorylated) p-Met was similar between B6 and TPR-Met mice (I-P). p-Met was not detected in control retina of B6 mice (I), but p-Met was detected as early as 3-6 hr after the laser injury. Expression of p-Met in the laser-treated areas obviously diminished (J) on day 3 and was almost undetected from day 7 to day 14 (K-L). Several p-Met positive migrating cells were observed after day 7 (K-L). p-Met expression was found in the control retina of TPR-Met mice (M) and dramatically increased on day 1 after the laser injury (N-P). Some migrated cells were detected from day 7 to day 14 (O-P). Expression of p-Met in TPR-Met mice after laser treatment was higher than in B6 mice (I-P). Expression of RPE65 in B6 mice on day 7 after laser treatment was significantly higher than other conditions (S), but there was no difference between the control, day 3 and day 14 (Q-R, T). In TPR-Met mice, RPE65 expression slightly decreased after laser injury on day 1 compared to the control (U-V), but significantly increased from day 7 to day 14 (W-X). The RPE layer in B6 and TPR-Met mice showed disorganized morphology after laser burns up to day 3, but started to reform on day 7, and completely reformed on day 14. The expression of c-Met in B6 and TPR-Met mice rapidly increased after the laser injury (Y). However, p-Met did not show obvious changes after the laser burns (Z). Expression of RPE65 in laser-treated B6 and TPR-Met mice was quite similar between B6 and TPR-Met mice at different time points (AA). The scale bar for all images: 100 μm. *P<0.05, independent samples t-test.

FIG. 6 (6A-6O) are a series of photomicrographs with bar graphs showing c-Met, p-Met and RPE65 expression in c-Met^{fl/fl} mice 14 days after AAV-GFP and AAV-Cre injections, respectively (A, C, E, G, I and K) without laser burns; and after laser burns (B, D, F, H, J and L). Mice were scarified on day 14 after laser application (total day, 28). In AAV-GFP injected mice, laser burns induced an expected increase in c-Met and p-Met (B vs. A; E vs. F). There was no detectable c-Met or p-Met expression seen after subretinal AAV-Cre injection (C and G). Less c-Met and no p-Met expressed were observed even after laser injury (D and H). RPE65 expression was not affected by subretinal injection of AAV-GFP or AAV-Cre injection (I-L). There were more migrated RPE cells in ONL in AAV-Cre injected mice (L) compared to AAV-GFP injected mice (J). There was very limited c-Met and p-Met detected in AAV-Cre injected eyes before and after laser injury (M-N); in AAV-GFP injected eyes, c-Met expression expectedly increased after laser treatment (M) (*P<0.05, independent samples t-test). RPE65 expression was not affected by either AAV-GFP or AAV-Cre injection (O).

FIG. 7 (7A-7H) are a series of photomicrographs and graphs showing the migration of RPE cells into the outer retina 7 days after injury in B6 mice. RPE cells were observed to migrate into the ONL and expressed both c-Met and p-Met (A-E); RPE65 expression confirmed that migrating cells were indeed RPE (C and F). RPE migration was observed as early as 3 days after injury. More robust RPE migration was observed in TPR-Met mice (left side in G panel). In AAV-Cre injected mice, significantly fewer migrating cells were found compared with their AAV-GFP injected counterparts (right side in G panel, *P<0.05, independent samples t-test). These observations indicate that higher c-Met expression could induce more RPE cells to migrate. Significant linear association was confirmed between c-Met expression and the duration of laser injury, specifically from day 3 to day 14 in both B6 mice (y=−0.50x+0.11, R²=0.82) and TPR-Met mice (y=−0.16x+0.13, R²=0.52) (H). On day 14 after the laser injury, the expression of c-Met in both mice gradually decreased back to baseline (control level, open circles on the right side). But the process in B6 mice may be faster (slope value, −0.50) than in TPR-Met mice (slope value, −0.16). In addition, a significant linear regression was found between the concentration of c-Met and the number of migrated RPE cells in the cMet over-expressed TPR-Met mice (y=0.83x+1.02, R²=0.62) (inserted graph in H panel, the values on x-axis and y-axis were log₁₀-transformed according to the original measurements). These findings strongly suggest that higher levels of c-Met expression could induce more RPE cells to migrate. The scale bar for all images: 100 μm.

FIG. 8 is a graph showing the expression of HGF and c-Met and the migration of RPE cells in B6 mouse after the laser-induced injury. The accumulation of HGF expression is believed to be able to trigger the expression of c-Met. Meanwhile, the c-Met expression positively affected the RPE cell migration after the laser injury.

### DETAILED DESCRIPTION OF THE EMBODIMENTS

Unless defined otherwise, all technical and scientific terms used herein have the same meanings as commonly understood by one of ordinary skill in the art to which this invention pertains. Although methods and materials similar or equivalent to those described herein can be used in the practice or testing of the present invention, the preferred methods, devices, and materials are now described. All technical and patent publications cited herein are incorporated herein by reference in their entirety.

The practice of the present invention will employ, unless otherwise indicated, conventional techniques of tissue culture, immunology, molecular biology, microbiology, cell biology and recombinant DNA, flow cytometry and cell sorting which are within the skill of the art. See, e.g., Sambrook and Russell eds. (2001) Molecular Cloning: A Laboratory Manual, 3^{rd} edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5^{th} edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology. See, also, Yuan et al., Plos One, 6(3), e17540, March 2011.

All numerical designations, e.g., pH, temperature, time, concentration, and molecular weight, including ranges, are approximations which are varied (+) or (−) by increments of 1.0 or 0.1, as appropriate. It is to be understood, although not always explicitly stated, that all numerical designations are preceded by the term “about”. It also is to be understood, although not always explicitly stated, that the reagents described herein are merely exemplary and that equivalents of such are known in the art.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above.

As used in the specification and claims, the singular form “a”, “an” and “the” include plural references unless the context clearly dictates otherwise. For example, the term “a pharmaceutically acceptable carrier” includes a plurality of pharmaceutically acceptable carriers, including mixtures thereof.

As used herein, the term “comprising” is intended to mean that the compositions and methods include the recited elements, but do not exclude others. “Consisting essentially of” when used to define compositions and methods, shall mean excluding other elements of any essential significance to the combination for the intended use. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transitional terms are within the scope of this invention.

A “host” or “patient” of this invention is an animal such as a mammal, or a human. Non-human animals subject to diagnosis or treatment are those in need of treatment such as for example, simians, murines, such as, rats, mice, canines, such as dogs, leporids, such as rabbits, livestock, sport animals, and pets.

The term “isolated” means separated from constituents, cellular and otherwise, in which the cell, tissue, polynucleotide, peptide, polypeptide, protein, antibody or fragment(s) thereof, which are normally associated in nature. For example, an isolated polynucleotide is separated from the 3′ and 5′ contiguous nucleotides with which it is normally associated in its native or natural environment, e.g., on the chromosome. As is apparent to those of skill in the art, a non-naturally occurring polynucleotide, peptide, polypeptide, protein, antibody or fragment(s) thereof, does not require “isolation” to distinguish it from its naturally occurring counterpart. An isolated cell is a cell that is separated form tissue or cells of dissimilar phenotype or genotype.

The term “laser” as used herein designates a device that emits light through a process of optical amplification based on the stimulated emission of electromagnetic radiation. The term “laser” is actually an acronym for “Light Amplification by Stimulated Emission of Radiation”. Lasers differ from other light sources because they emit light coherently. This spatial coherence allows a laser to be focused to a tight spot, thereby enabling applications like laser cutting and laser lithography. In addition to spatial coherence, lasers also have high temporal coherence which permits emission in a very narrow spectrum, i.e. lasers only emit a single color of light. Temporal coherence also allows lasers to emit pulses of light lasting only a femtosecond. There are several types of lasers such as gas lasers, chemical lasers, excimer lasers, solid-state lasers, fiber lasers, photonic lasers, semiconductor lasers, dye lasers, free-electron lasers and bio lasers. Solid-state lasers typically use a crystalline or glass rod which is doped with ions that provide the required energy states. Neodymium is a common dopant in solid-state laser crystals including yttrium aluminum garnet (Nd:YAG) lasers. Lasers currently have significant military and civilian applications.

As used herein, the terms “treating,” “treatment” and the like are used herein to mean obtaining a desired pharmacologic and/or physiologic effect. The effect can be prophylactic in terms of completely or partially preventing a disorder or sign or symptom thereof, and/or can be therapeutic in terms of a partial or complete cure for a disorder and/or adverse effect attributable to the disorder. Examples of “treatment” include but are not limited to: preventing a disorder from occurring in a subject that may be predisposed to a disorder, but has not yet been diagnosed as having it; inhibiting a disorder, i.e., arresting its development; and/or relieving or ameliorating the symptoms of disorder. As is understood by those skilled in the art, “treatment” can include systemic amelioration of the symptoms associated with the pathology and/or a delay in onset of symptoms such as chest pain. Clinical and sub-clinical evidence of “treatment” will vary with the pathology, the individual and the treatment. The treatments described herein can be used as stand alone therapies, or in conjunction with other therapeutic treatments.

A “composition” is intended to mean a combination of active agent, cell or population of cells and another compound or composition, inert (for example, a detectable agent or label) or active.

A “pharmaceutical composition” is intended to include the combination of an active agent with a carrier, inert or active such as a biocompatible scaffold, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin, Remington's Pharm. Sci., 15th Ed. (Mack Publ. Co., Easton (1975)).

An “effective amount” is an amount sufficient to effect beneficial or desired results. An effective amount can be administered in one or more administrations, applications or dosages.

### Laser Injury Model of Retinal Damage

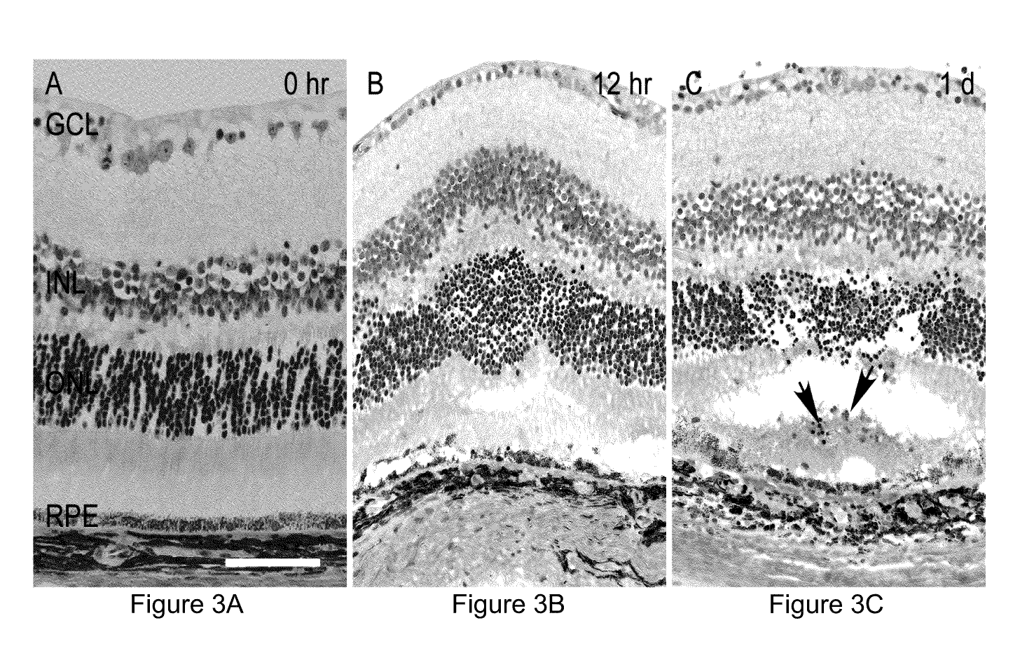

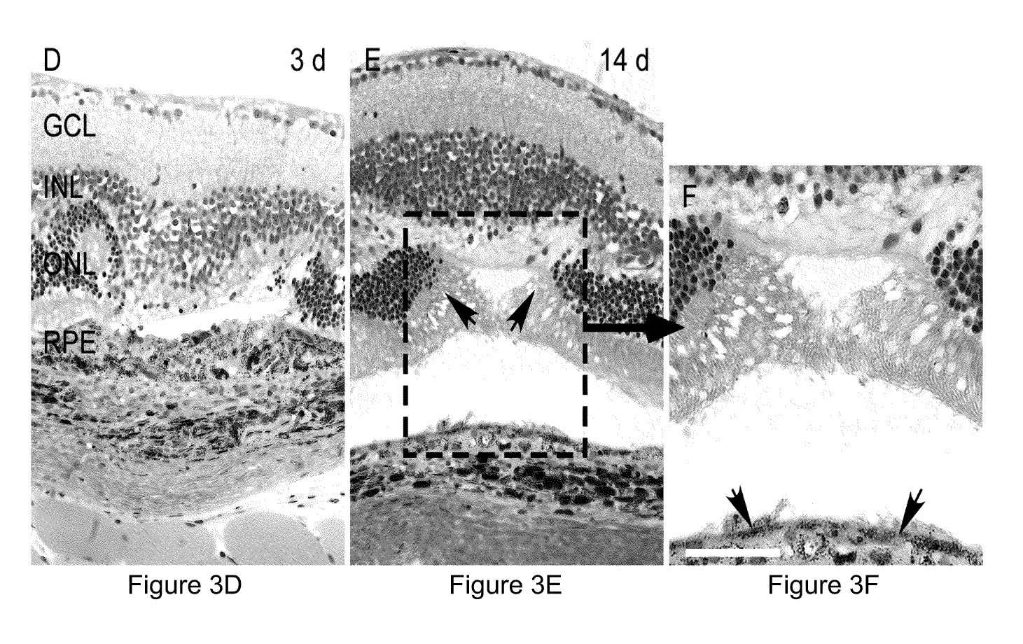

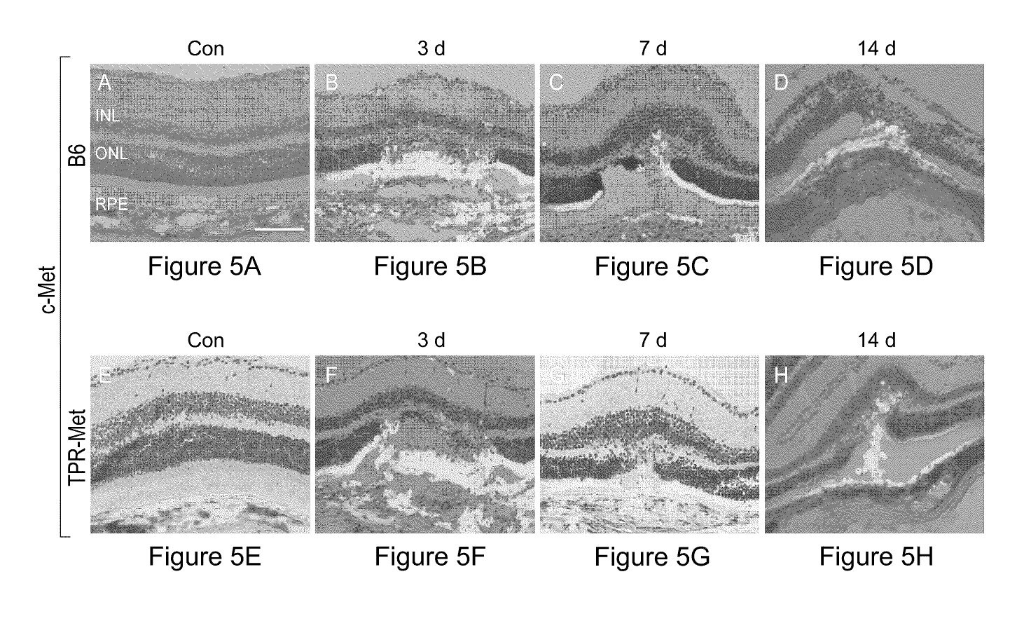

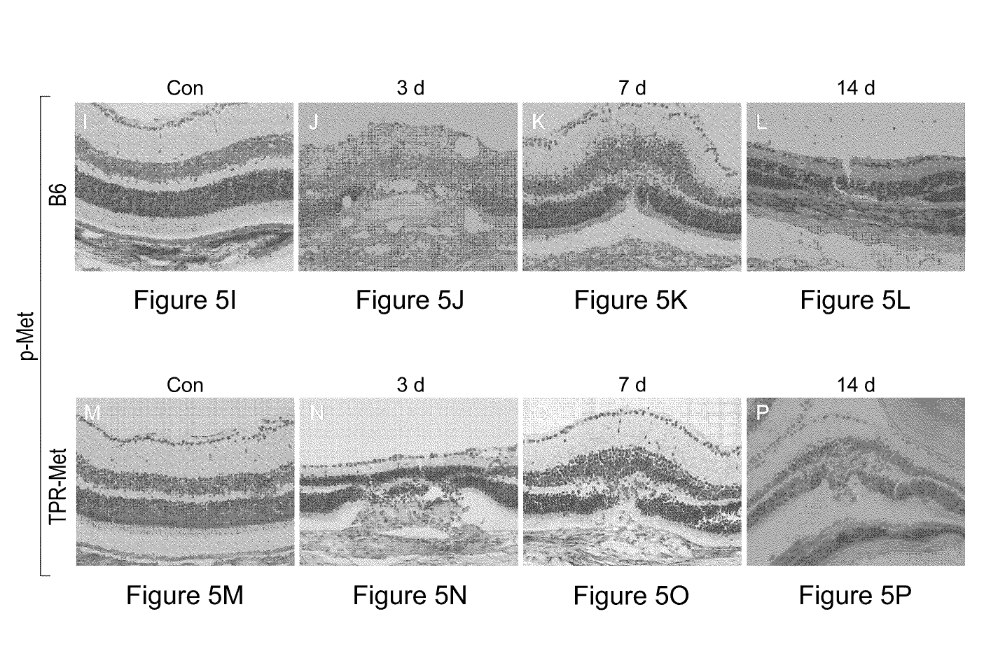

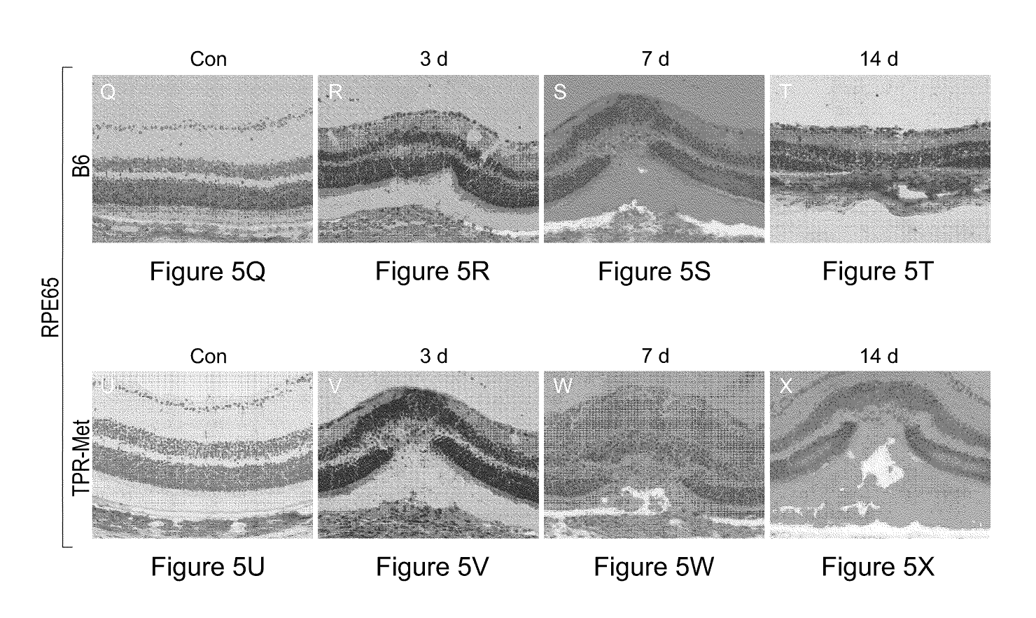

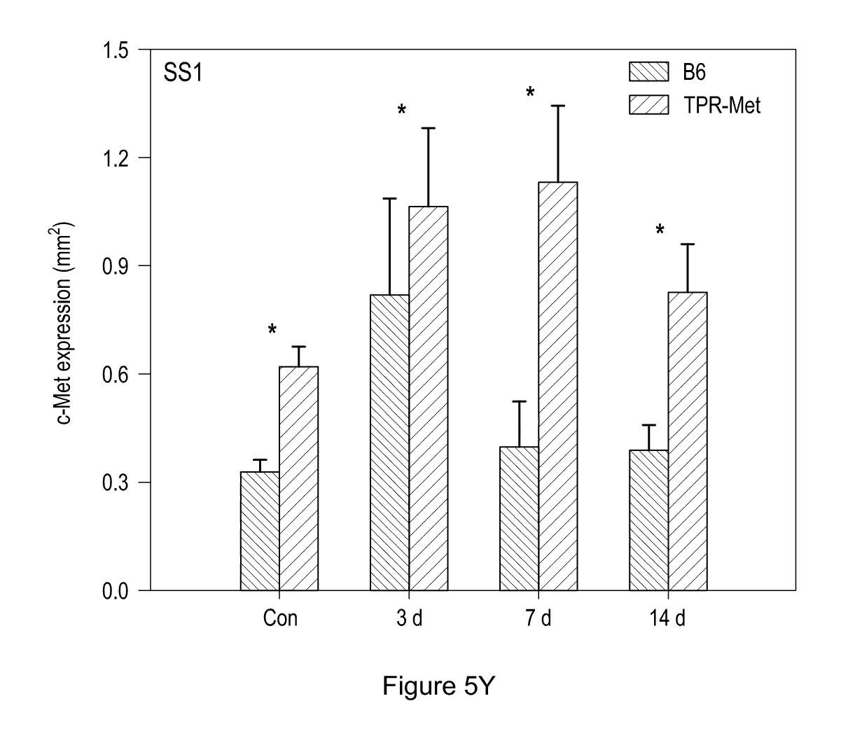

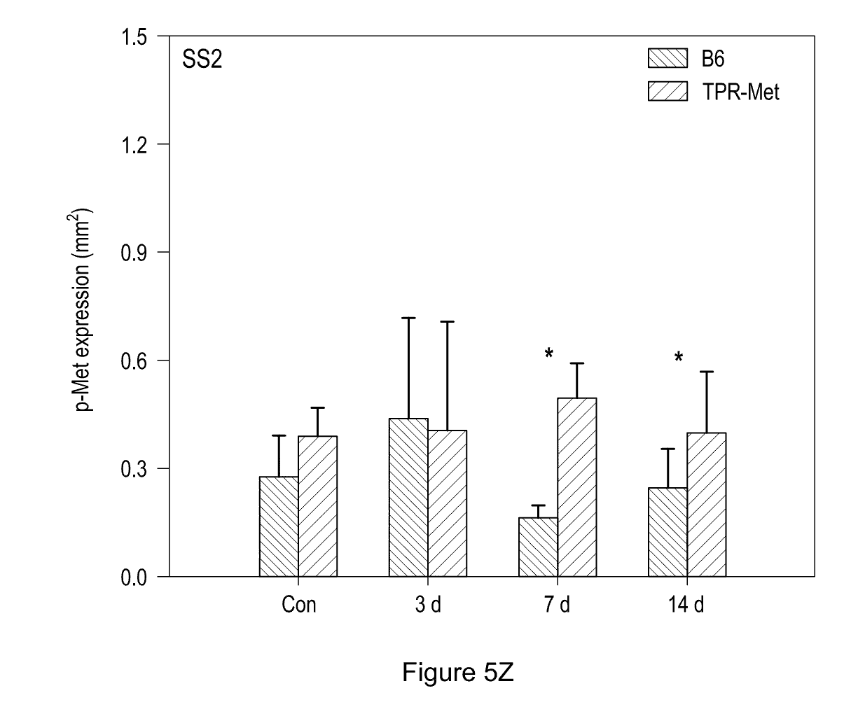

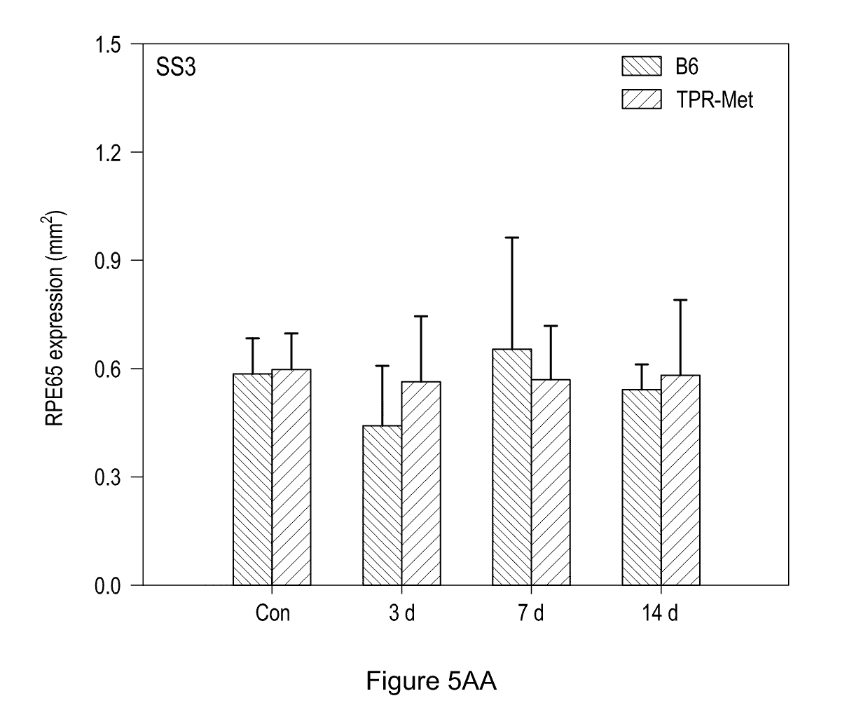

Laser-induced retinal alternations characterized to date indicate that with increased energy, damaged areas extend to outer segment layers of the retina in addition to the RPE, which itself is considered the primary site of absorption. These injuries mostly damage the RPE layer by photothermal and photodisruptive mechanisms (see FIG. 3C). The RPE layer begins to disorganize as early as 12 hr after the laser injury. The disorganization becomes quite severe on day 1 post-injury (FIG. 3). After the disorganization in the RPE layer, some RPE cells migrate toward the ONL, as confirmed by IHC (FIGS. 5-7). Visual loss after laser injury is related to the location of the laser damage. For example, laser injury to the fovea would likely lead to an immediate and significant vision loss. Parafoveal laser lesion may involve the fovea temporarily through inflammation and edema, resolving over days to weeks, or may spread to the fovea through secondary neuronal cell damage (creep), causing permanent defects.

### RPE Cell Migration and Proliferation

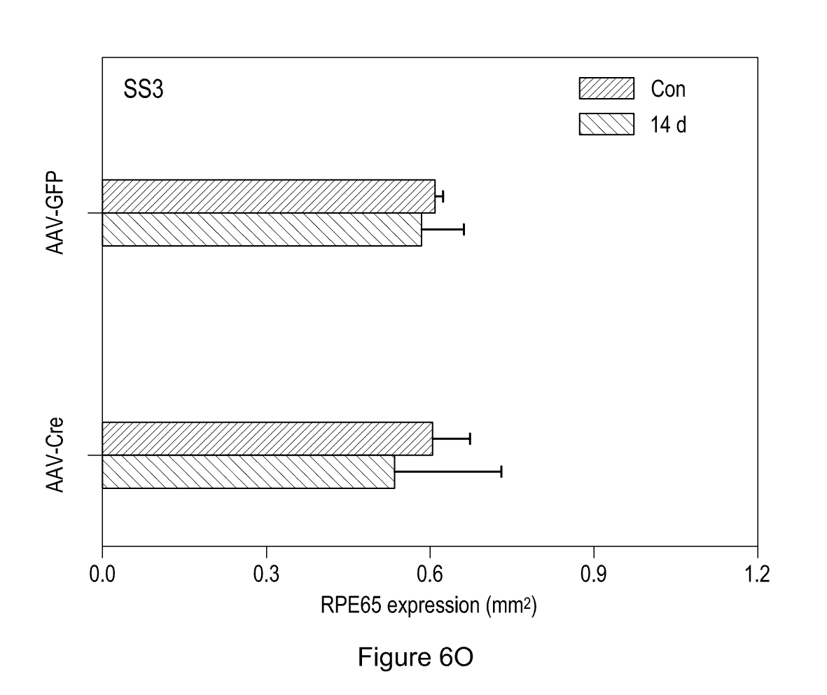

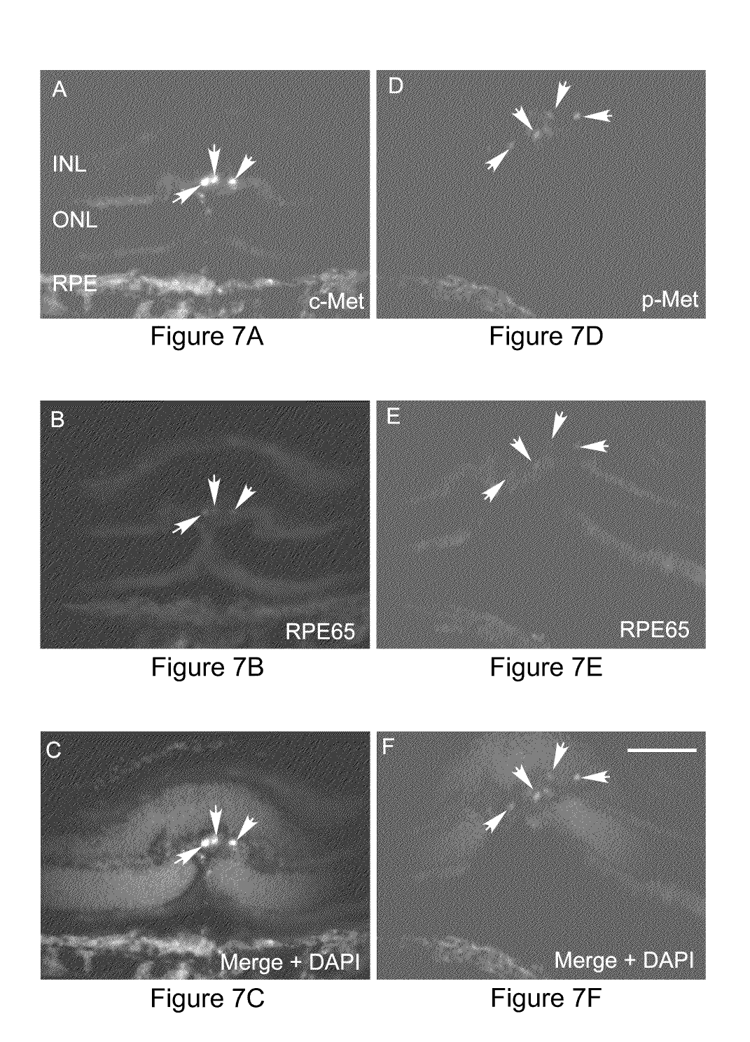

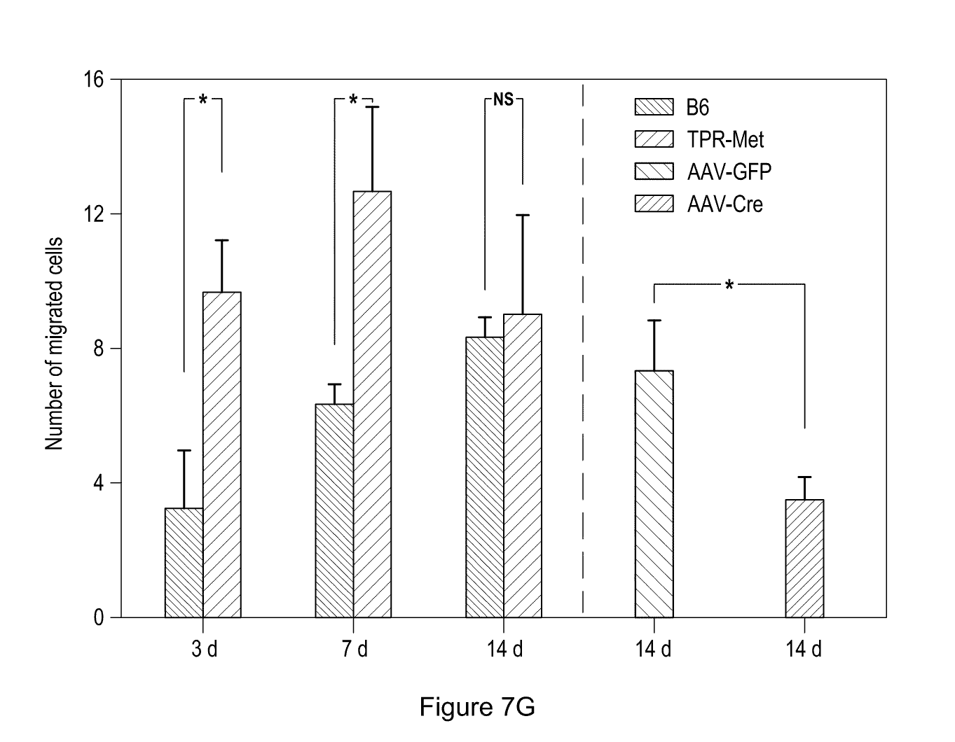

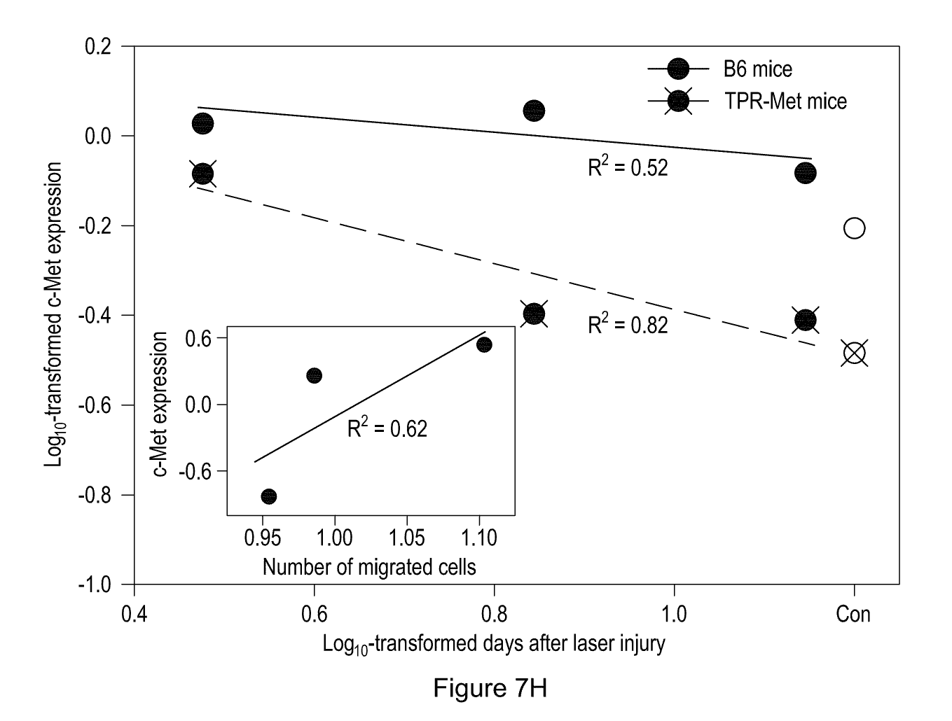

RPE cell migration and proliferation are believed to play a role in expansion of laser scars and the pathogenesis of PVR. Retinal injuries from laser exposure can have variable but potentially devastating effects, ranging from mild discomfort and dazzling to scaring and complete loss of central vision. The direct effect of these injures can not only lead to loss of photoreceptors and other neuronal cell types, but also to aberrant scar formation in the retina. The photoreceptors are largely lost from day 1 after the laser injury (FIGS. 3 and 7). Very frequently, parafoveal scars caused by laser injury expand to include previously uninvolved areas primarily due to aberrant RPE migration. If this migration and its ensuing scar formation involve the foveal center, central visual loss will ensue. Any approach to limiting RPE migration through receptor abrogation or inhibition of migratory mechanisms may potentially limit this damage.

Human vitreous contains not only mitogens for RPE cells but also factors that mediate their migration. Clinically, the appearance of RPE cells in the vitreous may be a consequence of injury or rhegmatogenous retinal detachment in which these cells now become exposed to the vitreous. However, RPE cells do not proliferate in the vitreous unless there is a break in the blood-ocular barrier that would allow serum including albumin and other factors to access the vitreous. Extremely low levels of coherent radiation can produce ultrastructural alterations in sensory retina without apparent change in the RPE. More severe injuries, such as those caused by Nd:YAG lasers, can induce the migration of RPE cells when the blood vessels are broken to cause serum leakage. In this setting, RPE cells can be induced to transdifferentiate and migrate.

RPE cells can transdifferentiate to either neurons or lens cells in culture. There is evidence that the association of RPE cells with the retinal vasculature is an important step in transdifferentiation. Cells expressing RPE65 were found in ONL of B6 mice on day 7 after the laser injury (FIG. 7O), suggesting its RPE origin. In TPR-Met mice with continuatively active c-Met, more RPE65 positive cells were observed on days 7 and 14 after the laser burns compared to their B6 counterparts (FIGS. 7S-7T and 7W-7X). Previous studies found that vitreous from eyes treated with each of the above modalities caused significant stimulation of RPE migration while control vitreous and saline injected vitreous caused very limited RPE stimulation. It has now been found that constitutive activation of c-Met induced stronger RPE cell migration from the laser-induced injury site to the outer layer of the retina; similarly, abrogation of c-Met activity in c-Met^{fl/fl} floxed mice reduced RPE migration into the wounded sites.

Role of c-Met and its Responses to Retinal Laser Injury

c-Met participates in cell growth and migration during embryonic development, and plays a significant role in skin regeneration process. The c-Met protein also known as the HGF receptor encodes for a thyrosine kinase receptor which is activated by HGF. Receptor-type tyrosine kinases are important in regulating epithelial differentiation and morphogenesis, and HGF plays a significant role in developing several epithelial organs. Additionally, HGF-Met signal inhibitors may have important therapeutic value for the treatment of metastatic cancers. Moreover, c-Met is overexpressed in a variety of tumors in which it plays a central role in malignant transformation.

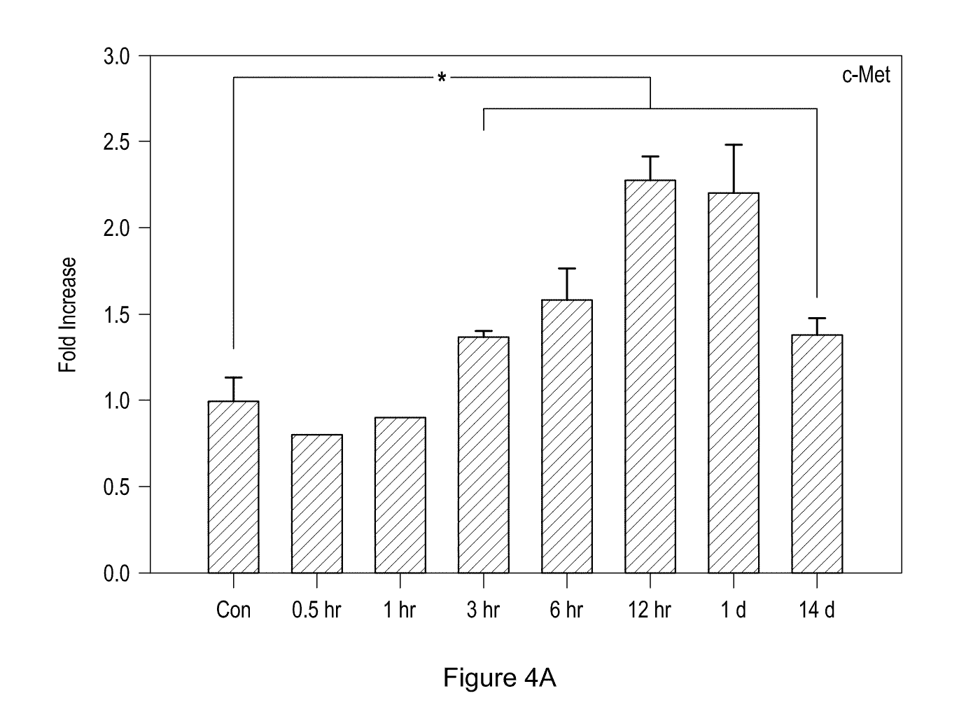

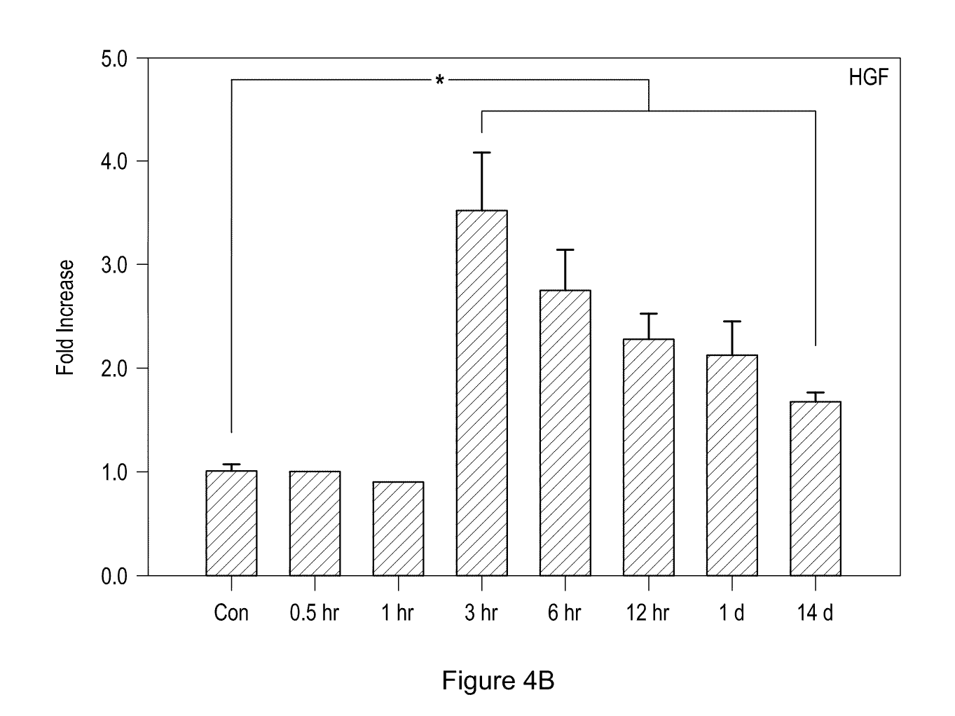

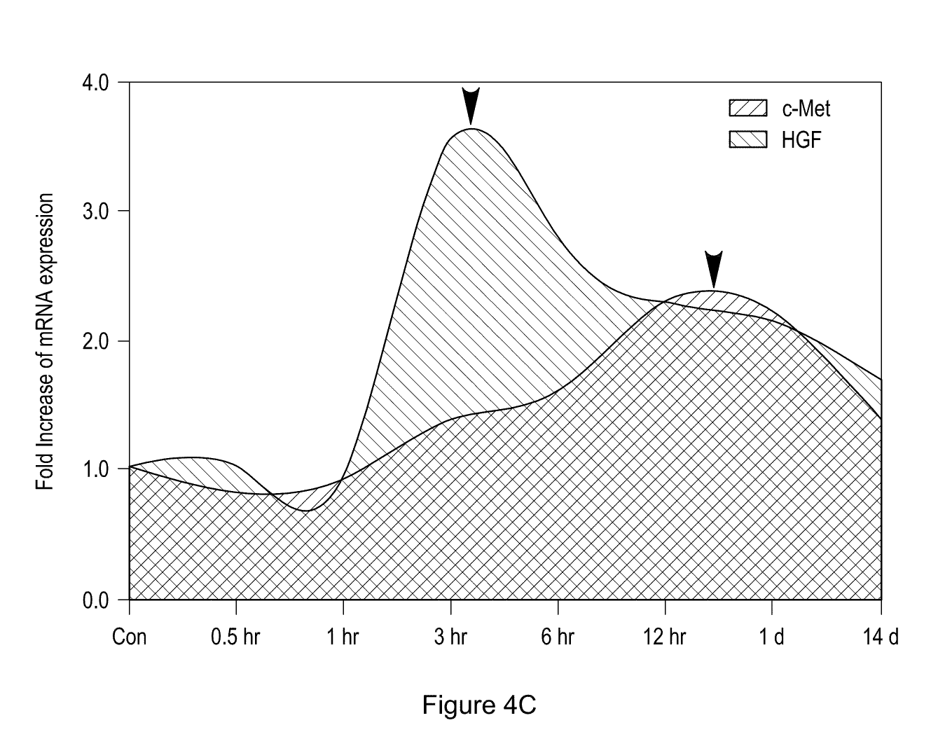

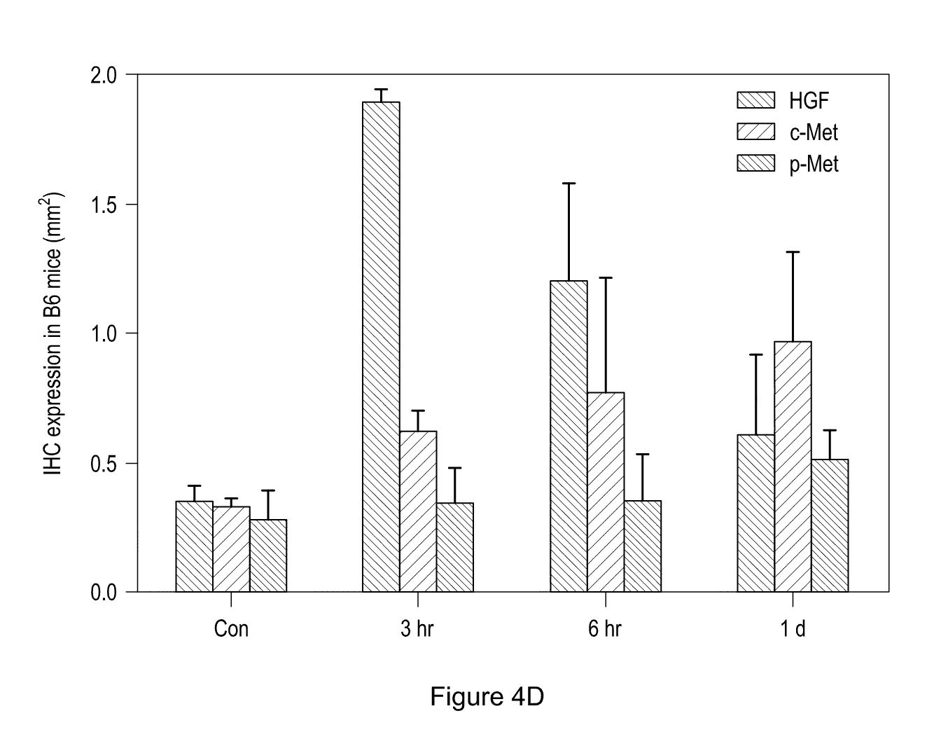

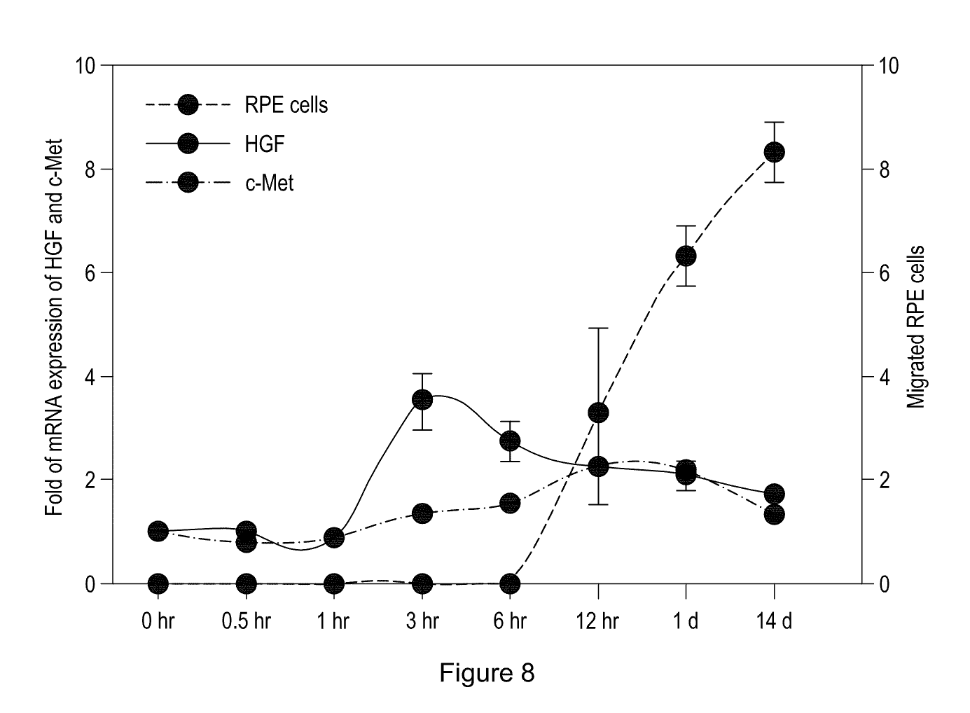

HGF is the only known ligand for the c-Met receptor. c-Met is normally expressed by cells of epithelial origin, while the expression of HGF is restricted to cells of mesenchymal origin. Upon HGF stimulation, c-Met induces several biological responses that collectively give rise to a program known as invasive growth. The accumulation of the HGF could initiate stimulation to the expression of c-Met. There was a hysteresis between the peak expressions of HGF and c-Met (FIGS. 4C and 8). This may indicate that as the receptor of HGF, the activation and expression of c-Met could only be triggered by a certain concentration of HGF.

The expression of cMet in constitutively activated TPR-Met mice was higher than in B6 mice (on days 3 and 7; see FIG. 5Y). Although the exact mechanism between c-Met expression and RPE cell migration is still unclear, the augmentation of c-Met expression could positively affect the migration of the RPE cells after the laser injury (FIG. 7H). In addition, in control mice (B6) the accumulation of c-Met expression probably leads to the increase in RPE cell migration after the laser injury (FIG. 8).

There are situations in vivo in which RPE cells may migrate, such as in development and wound healing (including PVR and in diseases such as age-related macular degeneration). After retinal laser injury, photon absorption primarily by the melanin pigment causes thermal damage to the retina. This absorbed laser light is densely concentrated in the RPE cell layer and focally absorbed in the choroid. This process may lead to the leakage of serum, which can significantly release HGF, activate c-Met receptors and induce migration of RPE cells. Activation of c-Met after laser injury may induce RPE cells to migrate and transdifferentiate. The two key observations that relate the role of c-Met on RPE cells to laser injury responses can be summarized as follows: (1) constitutive activation of c-Met via the TPR-met receptor increased both the expression of c-Met protein on RPE cells, and caused more robust RPE migration into outer retina, and (2) abrogation of the c-Met receptor using the Cre-Iox system reduced RPE migration without affecting RPE65 expression. Accordingly, and without being limited to any particular theory or hypothesis, limiting RPE migration is a critical factor to limit wound growth and creep, and consequently, the inhibition of c-Met activity is a viable method for limiting aberrant retinal wound responses.

### SUMMARY

Clinically, RPE cells can migrate anywhere in the retina. RPE cell migration may be mediated through the activation of the c-Met receptor. Accordingly, cMet activation induces transdifferentiation of RPE cells and its migration across the all retinal surfaces. In response to retinal laser injury, the c-Met receptor system is activated through the release of HGF, and is intimately involved in the responses of RPE to laser injury. This is supported by the observation that the constitutive activation of c-Met increased RPE migration into the retina, and abrogation of the receptor diminished RPE cell migration. Therefore, the control of c-Met activity is a viable therapeutic target to minimize retinal damage that may ensue after laser injury.

The invention may be further described and illustrated in the following examples which are not in tended to limit the scope of the invention thereby.

### EXAMPLES

### Materials and Methods

All experiments were performed in accordance with the association for Research in Vision and Ophthalmology Statement for the Use of Animals in Ophthalmic and Vision Research. Three different types of mice were compared as detailed in Table 1 below.

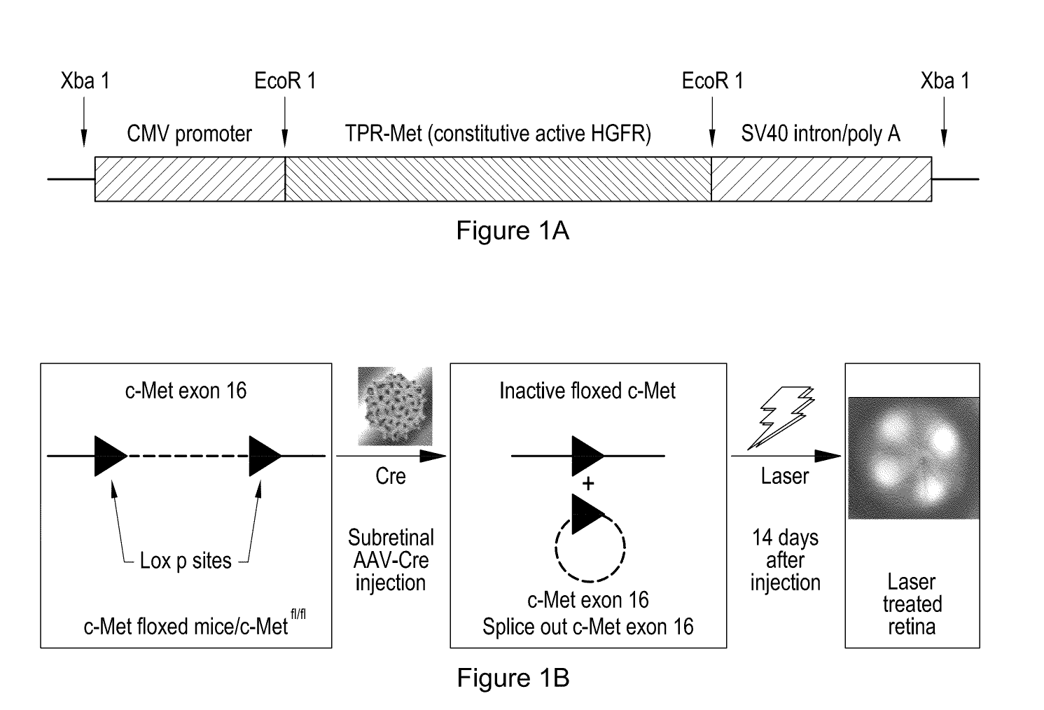

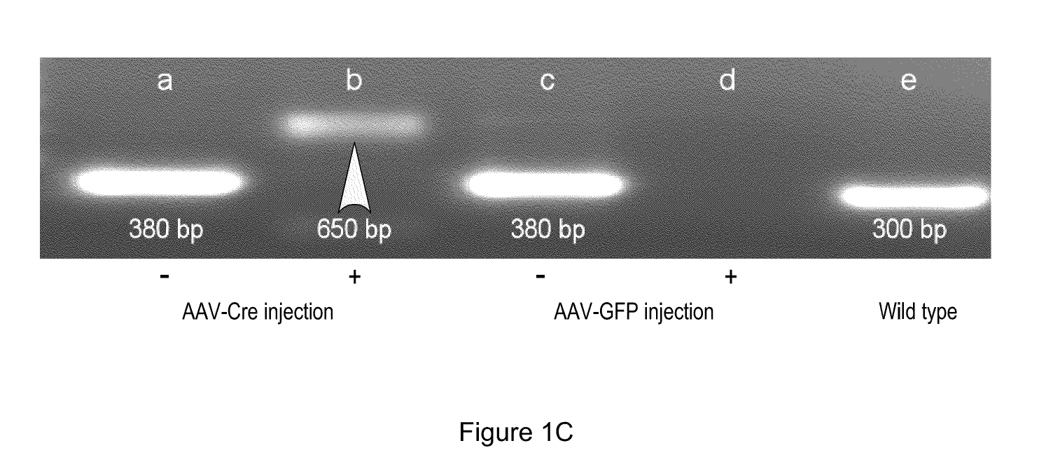

B57BL/6 (B6) mice were purchased from Charles River Laboratories (Cambridge, Mass.) and used as a model for wild-type c-Met expression. FVB/N-Tg/mtTPRmet mice were obtained from Jackson Laboratories (Bar Harbor, Me.), and backcrossed to B6 mice×6 to produce a stable colony (C57BL/6/FVB/N-Tg/mtTPRmet) in the B6 background (TPR-Met mice). In TPR-Met mice, the extracellular domain of c-Met gene was replaced with the TPR gene. This provided two strong demerization motifs and subsequent constitutive activation of the receptor in an HGF-independent manner. To ensure the proper c-Met expression in TPR-Met mice, SV40 splicing and polyadenylation signals were added to the structure (FIG. 1A). Heterozygous TPR-Met mice were used to evaluate the augmentation of cMet activity and identified by genotyping of their tail clippings.

To study the effects of c-Met receptor abrogation, 129-057BL/6-met^{fl/fl} mice, a conditional knockout of c-Met, was obtained from the National Cancer Institute (Bethesda, Md.). These mice were also backcrossed to B6 mice×6 to produce a stable colony in the B6 background (c-Met^{fl/fl} In c-Met^{fl/fl} mice, exon 16 of the c-Met genome is flanked by lox p sites. In the presence of Cre recombinase (delivered by subretinal injection of adeno-associated virus harboring Cre recombinase (AAV-Cre)), floxed p sites are permanently spliced out rendering c-Met inactive (FIG. 1B). To evaluate the efficiency of Cre-ligation of floxed p sites in the c-Met, retinal lysates of c-Met^{fl/fl} mice were prepared and subjected to PCR reaction, which produced a 380 bp amplification fragment specific to the floxed allele (FIG. 1C, a and c), or a 300 bp fragment specific to wild-type allele (FIG. 1C, e). Subretinal injection of AAV-Cre in the homozygous c-Met^{fl/fl} mice produced a 650 bp fragment specific to the deleted allele (FIG. 1C, b). In contrast, no such 650 bp fragment was detected after subretinal injection of AAV expressing green fluorescent protein (AAV-GFP) (FIG. 1C, d). The genotype of c-Met^{fl/fl} mice is summarized in the bottom panel in FIG. 1, and homozygous mice were used to evaluate the abrogation of c-Met by subretinal injection of AAV-Cre.

TABLE 1

| Mice | Background | c-Met Genome | c-Met Activity |
|---|---|---|---|
| B6 | C57BL/6 | Homozygous c-Met | Wild-type |
| | | receptor | |
| TPR-Met | C57BL6 × FVB/N- | Heterozygous | Constitutively |
| | Tg/mtTPRmet | TPRmet | active |
| c-Met | 129-C57BL/6-met^{fl/fl} × | Homozygous floxed | Wild-type; |
| | C57BL/6 | c-Met | abrogated by |
| | | | Cre |

### Procedure for Retinal Laser Injury

Mice were anesthetized with a mixture of Ketamine and Xylazine previously diluted in sterile saline at a dose of 120 mg/kg and 20 mg/kg, respectively. Only the right eyes of mice were used for laser treatment. The anesthesic mixture was injected intraperitoneally using a 27G needle. Mice were kept on a heat pad during and after the procedure of anesthesia. Pupils were dilated with topical application of 5% phenylephrine and 0.5% tropicamide solution. A flat glass cover slip was applied to the cornea to neutralize corneal and lenticular diopteric power. Laser burns were created using a diode laser (IRIS Medical OcuLight SLx, IRIDEX Corporation, Mountain View, Calif.) with a wavelength of 810 nm, spot size of 350 μm, 150 mW power for 150 ms. 12 laser spots were applied in each animal, 3 per each retinal quadrant centered around the optic nerve (FIG. 1B). For sham injections, the laser was set to “standby” and the foot pedal was depressed.

All mice were scarified by carbon dioxide inhalation at the different intervals from 0.5 hr to 14 days after the laser treatment. Eyes were quickly enucleated and fixed in 4% paraformaldehyde for histological examination. Some fresh retinas were collected to extract the mRNA for c-Met and HGF expression.

### TUNEL, Pigment Bleaching and Immunohistochemistry

After fixing overnight in 4% paraformaldehyde, eyeballs were transferred in PBS buffer with 30% sucrose for one hour and processed for either paraffin or frozen sections at a thickness of 8 μm. Terminal deoxynucleotidyl transferase dUTP nick end labeling (TUNEL), a common method for detecting DNA fragmentation that results from apoptotic signaling cascades, was used to confirm areas of laser damage. To this end, cryosections of the retina of B6 mice were stained with in situ Cell Death Detection Kit (Roche, Mannheim Germany).

Paraffin sections for IHC staining were warmed overnight, deparaffinized with xylene, taken through serial alcohol dilutions and hydrated to distilled water. Sections were bleached for melanin pigment using an established protocol. Briefly, sections were oxidized by incubation in 0.25% aqueous potassium permanganate for 30 min, washed in distilled water and bleached in 5% oxalic acid until white. Sections were then washed in PBS and subjected to immunohistochemistry using the Vectastain ABC kit with the alkaline phosphatase method and resolved with Vector Red (Vector laboratories, Burlingame, Calif.). Primary antibodies included HGF (H-145, Santa Cruz Biotechnology, Santa Cruz, Calif.), c-Met (SP260, Santa Cruz Biotechnology), phospho-c-Met (07-810, Upstate Biotechnology, Temecula, Calif.) and RPE65 (MAB5428, Chemicon, Temecula, Calif.). Sections were examined under an IX51 Olympus inverted fluorescent microscope (Olympus Corporation, Tokyo, Japan) both under visible light and epifluorescence for better detection of the highly fluorescent rhodamine Vector Red pigment. For better visualization, a grey-scale fundus image was sandwiched with its corresponding fluorescence image, which was itself assigned an arbitrary color (green, blue or red).

Effect of Laser Injury on Expression of c-Met and HGF in B6 Mice

To determine whether laser injury affected expression of c-Met and HGF in the RPE monolayer and outer retina, laser-induced retinas of B6 mice were collected at the following time points, 0, 0.5, 1, 3, 6, 12 hr, 1 day and 14 days (5 individuals per group). Total mRNA was extracted using RNA 4 Aqueous kit (Ambion Inc., Austin, Tex.). Reverse transcriptase reaction was performed for each mRNA sample using Retroscript kit (Ambion Inc., Austin, Tex.). 1 μg of total mRNA was used as a template to synthesize first-strand complementary DNA (cDNA). RT-qPCR was performed with 3 independent repetitions using the API Prism 7900HT Sequence Detection system (Applied Biosystems, Foster city, CA) according to the instruction of SYBER Green PCR Master Mix (Applied Biosystems, Foster City, Calif.). The reaction program included 2 min at 50° C., 10 min at 95° C., 40 cycles for 15 s at 95° C. and 60 s at 60° C. The parameter threshold cycle was designed as the fractional cycle number at which the fluorescence signals were generated during each PCR cycle. c-Met and HGF expression was calculated from the standard curve; quantitative normalization in each sample was performed using the expression of the glyceraldehyde-3-phosphate dehydrogenase (GAPDH) as an internal control using the delta-delta method. Data were presented as fold change over control. The sequences of the primers are summarized in Table 2 below.

TABLE 2

| Genes | Primers | Sequences |
|---|---|---|
| HGF | Forward | 5′-TTCCCAGCTGGTCTATGGTC-3′ |
| | | (SEQ ID NO: 1) |
| | Reverse | 5′-TGGTGCTGACTGCATTTCTC-3′ |
| | | (SEQ ID NO: 2) |
| c-Met | Forward | 5′-ATGAAATCCACCCAACCAAA-3′ |
| | | (SEQ ID NO: 3) |
| | Reverse | 5′-TCTGAATTTGAGCGATGCTG-3′ |
| | | (SEQ ID NO: 4) |
| GAPDH | Forward | 5′-AACAGCAACTCCCACTCTTC-3′ |
| | | (SEQ ID NO: 5) |
| | Reverse | 5′-CCTCTCTTGCTCAGTGTCCT-3′ |
| | | (SEQ ID NO: 6) |

Subretinal Injection of Adeno-associated Virus (AAV) in Homozygous c-Met′ Mice

AAV-Cre and AAV-GFP (serotype 2) were constructed and supplied by the Harvard Gene Intiative Core (Boston, Mass.). AAV vectors were previously purified and titrated to about 1×10¹⁰ Tu/ml for both AAV-Cre and AAV-GFP. Homozygous c-Met^{fl/fl} mice were injected either with a mixture of AAV-Cre/AAV-GFP (ratio 9:1) or AAV-GFP using a trans-scleral approach into the subretinal space under direct observation. The small amount of AAV-GFP in the AAV-Cre/AAV-GFP mixture induced a low-level background green fluorescence that can be detected in vivo by epifluorescence microscopy.

To perform this injection, a silk suture was penetrated through the upper eyelid. The eyelid was gently retracted and the eyeball was projected out from the eye socket to improve exposure. A small O-ring was placed on the eye. The pupil was dilated with one drop of each 2.5% phenylephrine and 0.5% tropicamide. Gonak (Akorn, Inc., Buffalo Grove, Ill.) was applied over the O-ring to make an optical connection for visualization of the fundus. A sclerotomy (puncture hole) was made in the posterior portion on the wall of the eye using a 31G needle. A micro-glass pipette was used to deliver 2 μl of AAV solution through the sclerotomy into the subretinal space. Mice receiving subretinal injection of AAV-GFP served as controls for mice receiving AAV-Cre.

Analgesic buprenex (2 mg/kg) was administered subcutaneously to mice before the procedure and every 12 hr for 2 days. Antibiotic ophthalmic ointment (Vetropolycin, Pharmaderm, Melville, N.Y.) was applied to the eyes three times daily for 2-3 days postoperatively. Two weeks after subretinal injection, c-Met^{fl/fl} mice were subjected to laser treatment. The injected mice (5 individuals in each group) were scarified on day 14 after laser treatment and examined for the expression of c-Met, p-Met and RPE65 in the outer retina and RPE layer.

### Quantification on the IHC Staining, Cell Migration and Data Analysis

The respective expression areas (mm²) of c-Met, p-Met, HGF and RPE65 in the IHC stained slices were measured with ImageJ 1.46d (resolution at 300 pixels/mm). These included comparison of quantified measurements. Migrated cells in ONL and up to INL on the IHC stained sections were also manually counted using ImageJ. One-Way ANAVO, Mann-Whitney U test and independent samples t-test were used to compare differences in the expression of the aforementioned markers and the number of migrated cells. To establish linear regressions, log₁₀-transformation was performed to normalize data and applied to the expression of cMet, the days after the laser treatment and the number of migrated cells in B6 and TPR-Met mice. The data were presented as mean±SD. All specific analyses and regressions were performed in SigmaPlot 11.0. The level of significance was set at 0.05.

### Results

### Cell Apoptosis and Histological Changes Induced by Laser Burns

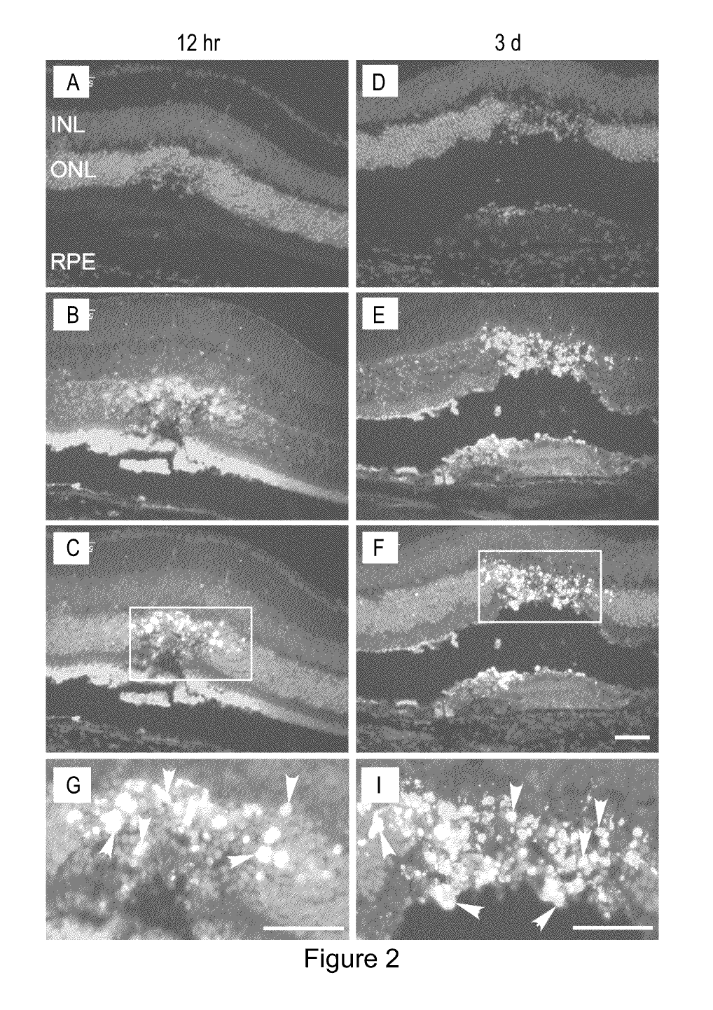

After laser injury, the retinal wound area appeared as creamy white spots (FIG. 1B, right side). Although no obvious morphological disorganization of retina was found at early stage after the laser burns (FIGS. 2A and 2D), apoptotic cells were detected in seemingly injured retinas. Apoptotic cells in ONL were detected by TUNEL as early as 12 hr after laser injury (FIGS. 2B-2C). On day 3, some dead cells were found in the RPE layer, and more dead cells were detected in the ONL (FIGS. 2E-2F). The typical apoptotic and dead cells were indicated by arrowheads (FIGS. 2G-2I). The RPE layer appeared intact before the laser burns (FIG. 3A). Laser burns disrupted the RPE monolayer resulting in aberrant migration of RPE cells to the ONL at the site of laser-induced injury. At 12-24 hr after laser burns, the ONL began to exhibit structural disorganization with some photoreceptor loss as well (FIGS. 3B-3C). On day 3, most of the photoreceptors had disappeared in the laser injured areas (FIG. 3D). No photoreceptors were observed in the injured areas on day 14 (indicated by arrows in FIG. 3E) where a scar had formed (FIG. 3F). However, RPE cells had settled down and aligned to form a new monolayer at the wounded area, suggesting that a new blood-retina barrier had reformed (indicated by arrows in FIG. 3F).

Quantified c-Met and HGF Expression in B6 Mice

The changes in the respective mRNA levels of c-Met and HGF in response to retinal laser burns were quantified in B6 mice using RT-qPCR. Mice received either sham or laser photocoagulation and were sacrificed at serial time points, ranging between 0.5 hr and 14 days. RT-qPCR results were presented as ratios of laser-treated and sham-treated retinas after being normalized to the expression of GAPDH.

There were no detectable changes in the respective expressions of c-Met and HGF mRNA in B6 mice in the first 3 hr after the laser injury (FIGS. 4A-4B). The mRNA level of c-Met reached its peak expression at 12-24 hr; on day 14, the mRNA level of c-Met was statistically higher than that of the control (all P<0.05, Mann-Whitney U test) (FIG. 4A). At 3 hr after the laser injury, the mRNA level of HGF dramatically increased, and gradually decreased thereafter, but remained significantly higher on day 14 as compared to sham-treatment (all P<0.05, MannWhitney U test) (FIG. 4B).

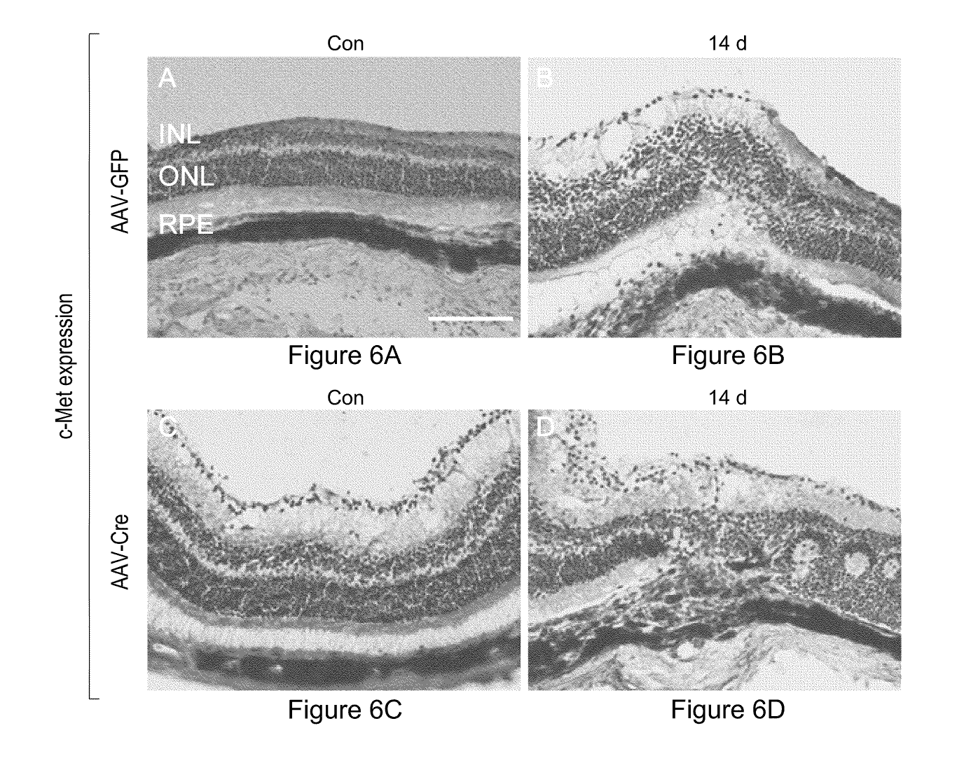

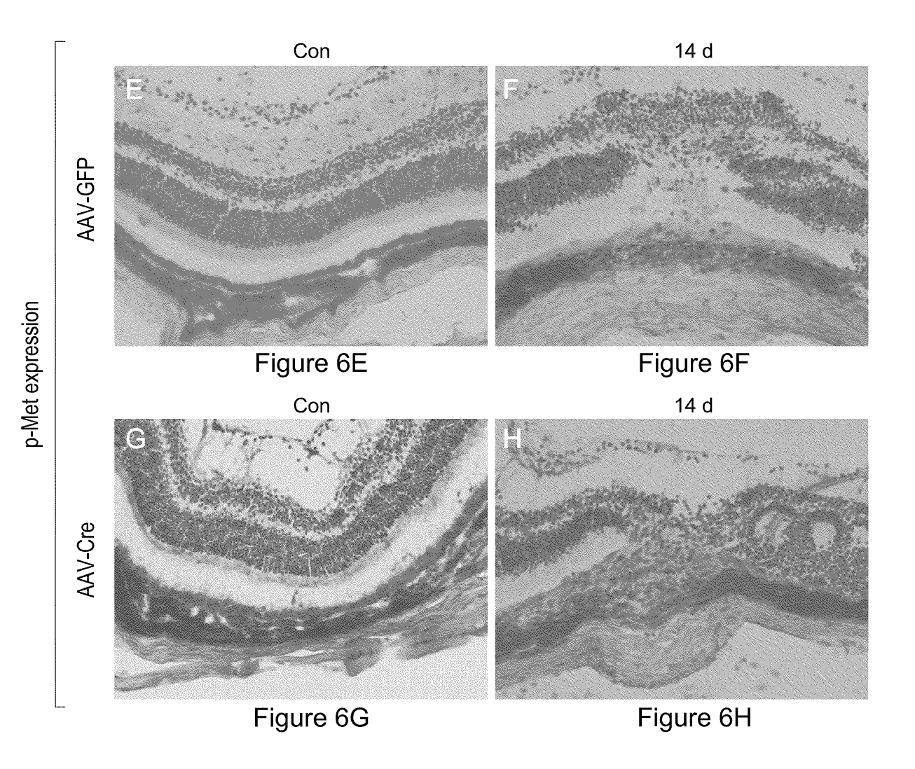

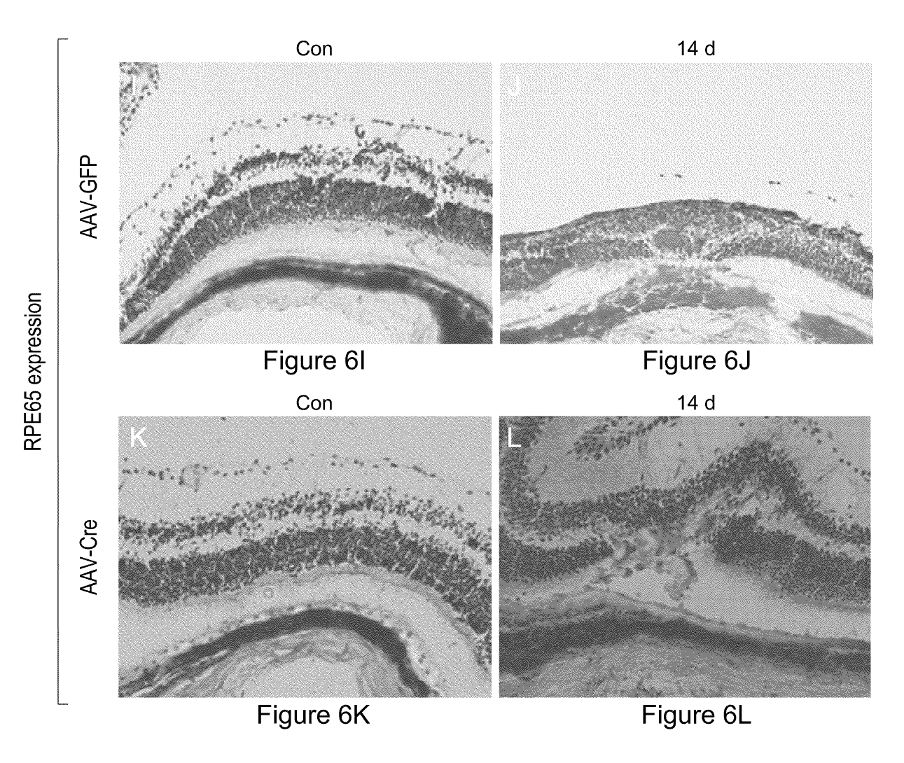

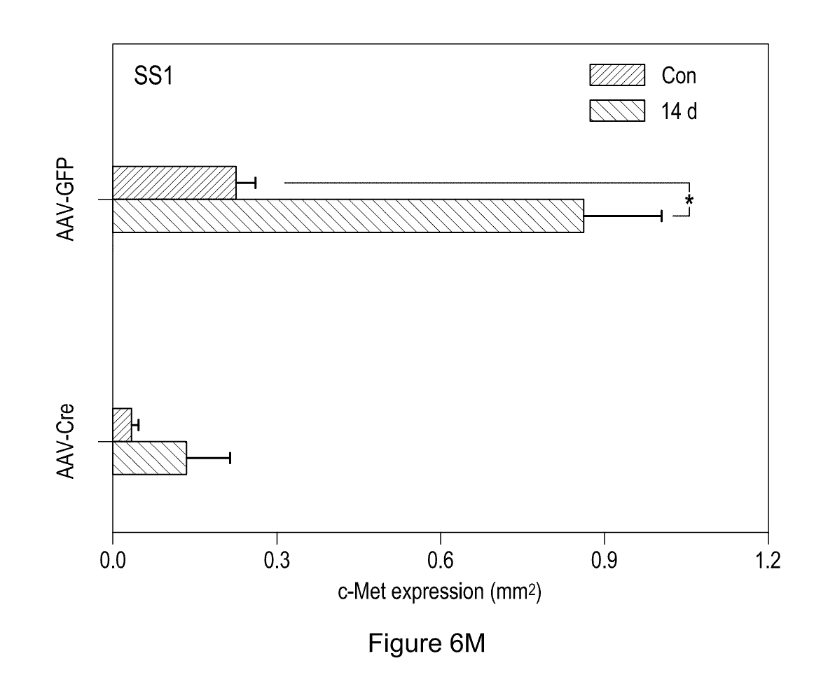

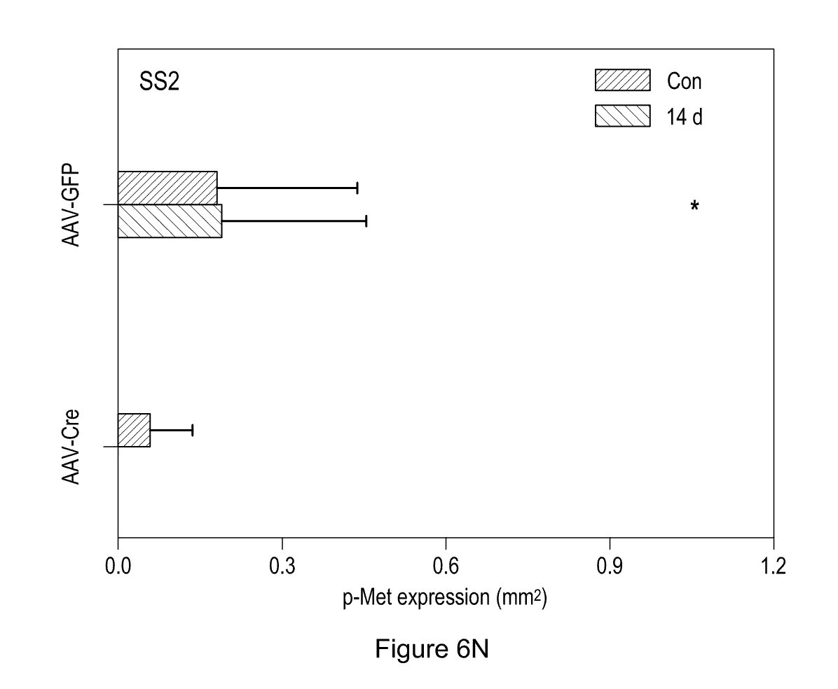

Interestingly, the mRNA level of c-Met did not show a simultaneous increase with the mRNA level of HGF. While HGF mRNA peaked at 3 hr after the laser injury, cMet mRNA peaked at 12 hr (indicated by arrowheads in FIG. 4C, respectively). The mRNA levels of HGF and c-Met were similar between 12 and 24 hr, but HGF mRNA remained constantly higher than that of c-Met during the whole time period (FIG. 4C). The hysteristic phenomenon on the mRNA expression of these two genes may indicate that c-Met, a receptor for HGF, would not be triggered simultaneously by the expression of HGF. The accumulation of HGF mRNA may be necessary to trigger c-Met mRNA expression. This hysteristic phenomenon on the expression of c-Met and HGF was also confirmed by IHC staining (FIG. 4D). (More detailed IHC images are shown in FIGS. 5 and 6). ImageJ was applied to measure the area of marker expression (c-Met, HGF and p-Met) on stained sections. These measurements indicate that HGF protein expression rapidly increased (1.90±0.05 mm²) and was significantly higher than the expression of cMet or p-Met (Independent samples t-test, all P<0.05) at 3 hr after the laser injury. c-Met protein expression responded much slower and peaked at one day after injury (0.97±0.35 mm²). The expression of p-Met was constant within the first 24 hr (range: 0.28-0.51 mm²) after laser injury (independent samples t-test, all P<0.05) (FIG. 4D).

Expression of c-Met, p-Met and RPE65 in 86 and TPR-Met Mice

Expression of c-Met, p-Met and RPE65 showed dynamic changes in the retinas of B6 and TPR-Met mice at different time points after laser injury. In B6 mice, very limited c-Met expression (0.33±0.03 mm²) was detected in the control retina (FIG. 5A). c-Met expression was higher in TPR-Met mice (0.62±0.05 mm²) (FIG. 5E). After laser application, c-Met expression increased up to day 3 (0.82±0.27 mm²) (FIG. 5B). On day 7 and 14, its expression (0.46±0.91 mm² and 0.30±0.04 mm², FIGS. 5C-D) decreased to near baseline and sham-lasered levels (independent sample t-test, both P>0.05). The increase of c-Met expression (FIGS. 5B-5D) was coincident with the RT-qPCR analysis (FIG. 4A).

In TPR-Met mice, c-Met expression significantly increased 3 days after laser injury (FIG. 5F) compared to the control (1.06±0.22 mm² vs. 0.62±0.05 mm², independent samples t-test, P<0.05, FIG. 5E). The c-Met expression of TPR-Met mice began to decrease from day 7 to day 14 (1.13±0.22 mm² and 0.83±0.13 mm², FIGS. 5G-H), but was still higher than the control (independent samples t-test, both P<0.05, FIG. 5E). Generally, the expression of c-Met in TPR-Met mice was higher than in B6 mice (independent samples t-test, P<0.05, FIG. 5Y). Prior to laser injury, there was low but detectable p-Met expression in untreated retinas of TPR-Met mice (0.39±0.08 mm², FIG. 5M) and significantly less in B6 mice (0.28±0.11 mm², FIG. SI). After laser injury, the expression of p-Met in B6 mice was subtly increased (0.44±0.28 mm², FIG. 5J) on day 3 and then returned to control levels from days 7 to day 14 in 86 mice (0.17±0.04 mm² and 0.25±0.11 mm², FIGS. 5K-5L). In TPR-met mice, p-Met expression on day 3 after laser injury (range: 0.41±0.40 mm²) was quite similar to control levels (0.39±0.08 mm²) (independent samples t-test, P>0.05, FIGS. 5M-5P). Quantified measurements of p-Met expression are presented in FIG. 5Z.

The expression of RPE65 in B86 mice after laser injury did not show any significantly changes among different time points (range: 0.44-0.65 mm², OneWay ANOVA, all P>0.05) (FIGS. 5Q-5T and FIG. 5AA). In TPR-Met mice, RPE65 expression was very stable after laser injury (0.56-0.58 mm²) compared to the control (0.60±0.10 mm², FIGS. 5U-5V), and no differences were found among them (One-Way ANOVA, all P>0.05, FIGS. 5U-5X and FIG. 5AA).

Responses of RPE Monolayer to Laser Injury in c-Met^{fl/fl} Mice

In this study, the mixture of 9:1 AAV-Cre/AAV-GFP or AAV-GFP was subretinaly injected into homozygous c-Met^{fl/fl} mice. One group of c-Met^{fl/fl} mice (n=5) received only AAV-GFP injection without laser burns (FIGS. 6A, 6E and 6I). In these AAV-GFP injected mice, both c-Met and RPE65 were detectable (0.23±0.04 mm² and 0.59±0.08 mm², respectively) and p-Met expression was very low (0.13±0.18 mm²). A second group of c-Met^{fl/fl} mice (n=5) received AAV-Cre/AAV-GFP (9:1) injection without laser burns. In these mice, no c-Met or p-Met was detected, but RPE65 was expressed normally (0.03±0.01 mm², 0.04±0.06 mm² and 0.61±0.07 mm², respectively) (FIGS. 6C, 6D and 6K). A third group of c-Met″″ mice received subretinal injection of AAV-GFP followed by laser injury (2 weeks later). 14 days after the laser injury, c-Met and p-Met expression of AAV-GFP injected mice increased comparing their control counterparts (0.86±0.14 mm² and 0.14±0.19 mm², respectively) (FIGS. 6B and 6F). However, RPE65 expression remained similar to controls (0.61±0.01 mm², 0.59±0.08 mm², respectively) (FIGS. 6I-6J). A fourth group of c-Met^{fl/fl} mice received subretinal injection of AAVCre/AAV-GFP (9:1) followed by laser injury (2 weeks later). In these mice, the expression of c-Met was very limited (0.13±0.08 mm²) (FIG. 6D) and no p-Met was detected (FIG. 6H). RPE65 expression was not affected (0.54±0.20 mm²) (FIG. 6L). Overall, laser injury in AAV-Cre injected c-Met^{fl/fl} mice induced a relative (statistically non-significant) increase in c-Met expression compared to controls (FIG. 6M) without any detectable p-Met expression (FIG. 6N) or changes in RPE65 expression (FIG. 6O).

Role of c-Met on RPE Cell Migration after Laser Burns

Cryosections of B6 mouse retina were used to count the number of migrated cells in response to the laser injury. On day 7 after injury, migrated cells were observed in the ONL. IHC confirmed that these cells were indeed RPE. Furthermore, it indicated that c-Met (FIG. 7A), p-Met (FIG. 7D) and RPE65 (FIGS. 7B and 7E) were detected in migrated cells. Co-staining also confirmed that the migrated cells originated from the RPE layer (FIGS. 7C-7F), and RPE65 was used to confirm that migrated cells were indeed RPE cells in the three types of mice.

For quantifying migrated RPE cells after laser injury, cells were manually counted on the IHC staining slices using ImageJ. In B6 mice, RPE cells were identified in the ONL on day 3 after the laser injury (3±2 cells), and more cells were identified on days 7 (6±1 cells) and 14 (8±1 cells) (FIG. 7G). In TPR-Met mice with laser injury, more migrated RPE cells (FIG. 7G) were observed on days 3 (10±2 cells) and 7 (13±3 cells) compared with B6 mice (independent samples t-test, both P<0.05, at left side of FIG. 7G). However, the number of migrated RPE cells in TPR-Met mice began to decrease (9±3 cells) on day 14; and this response was similar to B6 mice (independent samples t-test, P>0.05, FIG. 7G). In contrast, p-Met expressing migrated cells were quite rare in both B6 mice (3±1 cells) and TPR-Met mice (5±2 cells) (FIGS. 5P and 5L) after the laser treatment. In c-Met^{fl/fl} mice, few c-Met positive cells (4±1 cells) were found in ONL of AAV-Cre injected mice (FIGS. 6D and 7G), but more migrated cells (8±2 cells) were found in AAV-GFP injected mice (independent samples t-test, P=0.03) (FIGS. 6B and 7G). No p-Met positive migrated cells were detected after AAV-Cre injection and laser treatment (FIGS. 6F and 6H).

To investigate the temporal relationship for c-Met expression after laser injury in B6 and TPR-Met mice, log₁₀-transformation was applied to both the days and c-Met expression from day 3 to day 14. Linear regression analysis confirmed that c-Met expression was significantly negatively related to the time (days) after the laser treatment in B6 (y=−0.50x+0.11, R²=0.82) and TPR-Met mice (y=−0.16x+0.13, R²=0.52) (FIG. 7H). Furthermore, the tread line indicated that the change of c-Met expression in B6 mice more rapidly returned to control levels than in TPRMet mice (FIG. 7H). This observation suggests that laser injury is able to induce higher and longer c-Met expression in the TPR-met mice.

Interestingly, a significant linear relationship was confirmed between the expression of c-Met and the migration of RPE cells in TPR-Met mice (y=0.83x+1.02, R²=0.62, FIG. 7H). In other words, the migration of RPE cells was positively associated with the concentration of c-Met expression. Therefore, in TPR-Met mice, laser injury induced constitutively high c-Met expression, which induced more RPE cells to migrate to the inner layers of the retina. In contrast, the relative abrogation of c-Met expression in c-Met^{fl/fl} mice reduced RPE migration after laser injury.

From the foregoing, it will be appreciated that, although specific embodiments of the invention have been described herein for purposes of illustration, various modifications may be made without deviating from the spirit and scope of the invention as set forth in the appended claims. All publications, patents, and patent applications referenced herein are incorporated by reference in their entirety.

What is claimed is:

1. A method of reducing scar formation and vision loss in the retina of a mammal comprising:
administering to the mammal an effective amount of a composition that inhibits the activity of the c-Met receptor.

2. The method of claim 1 wherein the composition that inhibits the c-Met receptor activity is an antibody that binds to the c-Met receptor, or an antagonist to the c-Met receptor.

3. The method of claim 2 wherein the activity of the c-Met receptor is inhibited by interfering with the binding of c-Met to the ligand HGF.

4. The method of claim 1 wherein the composition is administered locally, topically, intraocularly, peribulbarly or intravitreally.

5. The method of claim 1 wherein the mammal is a human.

6. The method of claim 1 wherein the scar formation and vision loss results from the migration of RPE cells into the outer retina.

7. The method of claim 6 wherein the scar formation and vision loss is the result of penetrating or non-penetrating ocular trauma, retinal detachment resulting in the release of RPE cells, choroidal scar formation, or a laser selected from the group consisting of thermal lasers, Nd:YAG lasers, and non-thermal lasers, or any combination of the foregoing.

8. The method of claim 7 wherein the non-thermal laser is a therapeutic photodynamic laser.
